(19) United States
(12) Patent Application Publication
(10) Pub. No.: US 2023/0241083 A1
(43) Pub. Date: Aug. 3, 2023

(54) FORMULATION COMPRISING CANNABINOIDS

(71) Applicant: CANNOVEX BV, Diepenbeek (BE)

(72) Inventors: Bram BECKERS, Diepenbeek (BE); Steven PETERS, Diepenbeek (BE)

(21) Appl. No.: 18/002,190

(22) PCT Filed: Jun. 18, 2021
(86) PCT No.: PCT/EP2021/066643

§ 371 Date: Dec. 16, 2022

(30) Foreign Application Priority Data
Jun. 19, 2020 (EP) 20181179.1

(51) Int. Cl. A61K 31/00; A61K 9/70; A61K 45/06

(52) U.S. Cl. CPC A61K 31/658; A61K 9/7069; A61K 45/06

(57) ABSTRACT

The present invention relates to specific types of pharmaceutical formulations and compositions comprising cannabinoids for use in the treatment of various disorders.

### FIELD OF THE INVENTION

The present invention relates to specific types of pharmaceutical formulations and compositions comprising cannabinoids for use in the treatment of various disorders.

### BACKGROUND TO THE INVENTION

Research has shown various application domains for the use of cannabinoids over the past years. Therefore, the production of cannabinoid pharmaceuticals is gaining more and more attention. Although there is substantial evidence available for the therapeutic effects of cannabinoids, more research and clinical trials are of utmost importance in order to meet the demand of safe and efficient pharmaceutical formulations containing cannabinoids. These formulations could then provide for suitable alternatives in areas such as (secondary) pain, itch, secondary impetigos, swelling, inflammation and bacterial infection treatment.

The choice of suitable pharmaceutical formulations greatly depends on factors such as: drug absorption (rate), metabolization and bioavailability. Therefore, a pharmaceutical formulation should be chosen bearing in mind the properties of the active substances as well as the desired drug release. For example, when transdermal drug release is required, transdermal patches may offer a suitable candidate. These pharmaceutical formulations provide for direct entry of the active substances into the systemic circulation, bypassing the so-called first pass effect of the liver and as such increasing the overall bioavailability of the drug. Especially for those active substances which are deactivated considerably by the liver (e.g. a number of cannabinoids), transdermal patches have proven to be a valuable alternative to conventional oral drug therapy, such as capsules or tablets, as well as for molecules with short half-lives. Transdermal patches come in different varieties, such as e.g. reservoir/controlled-release membrane patches, matrix patches and drug-in-adhesive (DIA) patches. These can be used to provide for different rates of uptake of active substances once topically applied.

Furthermore, these transdermal patches are particularly suitable for use in the treatment of pain in general and the treatment of disorders characterized by blistering of the skin, such as Epidermolysis bullosa. Frequently applied treatments show great resemblance with partial-thickness burns treatments. In all of these cases, pain relief forms a main aspect which contributes to the elevation of the subject's comfort. These transdermal patches are also suitable for the treatment of burns due to for example heat, chemicals or radiation, ranging from first degree burns to third degree burns.

Different types of strong analgesics are often used to treat pain and these disorders (e.g. tramadol and opiates), which involve an inherent risk of long-term addiction and/or overdose. Because these disorders often require long-term treatment, these risks tend to increase over time and when using higher doses of active substances. Therefore, the need of alternatives having similar effects while reducing these risks remains high.

The present invention fulfils the need of a cannabinoid pharmaceutical formulation with a high bioavailability, by providing for novel transdermal patches comprising one or more cannabinoids being suitable for use in the treatment of disorders such as blisters, neurodegenerative disorders, post-traumatic stress syndrome and pain and the secondary symptoms caused by these disorders.

### SUMMARY OF THE INVENTION

The present invention relates to a combination comprising Tetrahydrocannabinol (THC) and Cannabidiol (CBD) for use in the treatment of a disorder selected from the list comprising: pain, blisters, neurodegenerative disorders or post-traumatic stress syndrome, wherein said combination is formulated in a transdermal patch, characterized in that said transdermal patch comprises at least one transdermal nanofibrous film; in particular at least one transdermal nanofibrous electrospun film.

In a following embodiment, said nanofibrous film comprises nano-encapsulated THC and/or CBD.

In a next embodiment, said combination is further characterized in that at least one of said THC and CBD is a prodrug.

In a next embodiment, said transdermal patch is selected from the list comprising: reservoir patch, matrix patch, vapor patch or drug-in-adhesive patch. Said drug-in-adhesive patch may be selected from the list comprising: single-layer drug-in-adhesive patch or multi-layer drug-in-adhesive patch.

In a further embodiment, the transdermal patch and/or film of the present invention may further comprise at least one polymer, such as polyethylene oxide.

Also, in some embodiments, said transdermal patch and/or film may further comprise a skin penetration enhancer (PE) also referred to as “skin permeation enhancer”, such as selected from the list comprising: isopropyl myristate, decyl oleate, oleyl alcohol, octyldodecanol, propylene glycol, triacetin, cocoyl caprylocaprat; alcohols such as short chain alcohols (Ethanol, Isopropyl alcohol); Long chain alcohols (Decanol, Hexanol, Lauryl alcohol, Myristyl alcohol, Octanol, Octyl dodecanol, Oleyl alcohol), amides such as Cyclic amides (azone), esters such as alkyl esters (Ethyl acetate); Benzoic acid esters (Octyl salicylate, Padimate 0); Fatty acid esters (Ethyl oleate, Glyceryl monoleate, Glyceryl monocaprate, Glyceryl tricaprylate Isopropyl myristate, Isopropyl palmitate, Propylene glycol monolaurate, Propylene glycol monocaprylate), Ether alcohols (Transcutol®), Fatty acids (Lauric acid, Linoleic acid, Linolenic acid, Myristic acid, Oleic acid, Palmitic acid, Stearic acid, Isostearic acid), Glycols (Dipropylene glycol, Propylene glycol, 1,2-butylene glycol, 1,3-butylene glycol), Pyrrolidones (N-methyl pyrrolidone, 2-pyrrolidone), Sulphoxides (Decylmethyl sulphoxide, Dimethyl sulphoxide), Surfactants such as Anionic surfactants (Sodium lauryl sulphate); Cationic surfactants (Alkyl dimethylbenzyl ammonium halides, Alkyl trimethyl ammonium halides Alkyl pyridinium halides); Non-ionic surfactants (Brij 36T, Tween 80), Terpenes such as Monoterpenes (Eugenol, d-Limonene, Menthol, Menthone) and Sesquiterpenes (Farnesol, Neridol).

In a further embodiment, said transdermal patch and/or film of the present invention may further comprise an antioxidant such as vitamin C (ascorbic acid), vitamin E (Tocophersolan; TGPS); uric acid, lipoic acid, glutathione.

In a next embodiment, the present invention discloses a combination as defined herein, for use in the treatment of a disorder selected from the list comprising: pain, blisters, neurodegenerative disorders or post-traumatic stress syndrome, wherein said skin blisters are caused by burns or other related traumas or a disorder selected from the list comprising: skin allergies, different forms of eczema, bullous pemphigoid, bullous impetigo, dermatitis herpetiformis, pemphigus vulgaris, mucous membrane pemphigoid, pemphigoid gestationis, epidermolysis bullosa, pemphigus foliaceous or toxic epidermal necrolysis.

In a further embodiment, said neurodegenerative disorder is selected from the list comprising: Alzheimer's disease (AD) and other dementias, Parkinson's disease (PD) and PD-related disorders, Essential Tremor, Multiple System Atrophy, Huntington's disease or Motor Neuron Diseases (MND).

In a next embodiment, the present invention provides a combination as defined herein for use in the treatment of a disorder selected from the list comprising: pain, blisters, neurodegenerative disorders or post-traumatic stress syndrome is disclosed, wherein said use includes the alleviation of secondary symptoms caused by said disorders, such as selected from the list comprising secondary pain, itch, secondary impetigos, swelling, inflammation or bacterial infection.

In another embodiment, the present invention provides a combination as defined herein for use in the treatment of a disorder selected from the list comprising: pain, blisters, neurodegenerative disorders or post-traumatic stress syndrome is disclosed, wherein said use includes the reduction of opioid consumption/dependency in the treatment of said disorders.

In another embodiment, the combination for use as defined herein may comprise one or more additional pharmaceutically active agents suitable for use in the treatment of said disorders.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Permeability of three transdermal nanofibrous films with 10 mg Δ9-THC as active pharmaceutical ingredient (API) prepared using electrospinning.

A Franz diffusion cell was used to test the in vitro permeation. In vitro permeability testing of the three different mixtures (CNV6, CNV11 and CNV12) resulted in a permeability ranging from 9-13% after 48 hours.

FIG. 2: Stability study of THC in a receiver solution.

The THC concentration (ng/mL) does not decay over time (3, 6, 8, 24, 30, 48 hours) in a receiver solution EtOH:H₂O (50/50 vol).

### DETAILED DESCRIPTION OF THE INVENTION

The present invention will be described with respect to particular embodiments and with reference to certain drawings, but the invention is not limited thereto. The drawings, as further described, are only schematic and non-limiting.

Furthermore, the terms first, second, further and the like in the description and in the claims are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a product comprising A and B” should not be limited to products consisting only of elements A and B. It means that, with respect to the present invention, the relevant elements of the product are A and B and that further components such as C may be present.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this description, with each claim standing on its own as a separate embodiment of this invention.

Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details.

In other instances, well-known methods, structures and techniques have not been shown in detail in order not to obscure an understanding of this description.

As already detailed herein before, in a first aspect, the present invention relates to a combination comprising Tetrahydrocannabinol (THC) and Cannabidiol (CBD) for use in the treatment of a disorder selected from the list comprising: pain, blisters, neurodegenerative disorders or post-traumatic stress syndrome, wherein said combination is formulated in a transdermal patch, characterized in that said transdermal patch comprises at least one transdermal nanofibrous film; in particular at least one transdermal nanofibrous electrospun film.

Alternatively, the present invention relates to a combination of one or more cannabinoids or derivatives thereof for use in the treatment of a disorder selected from the list comprising: pain, blisters, neurodegenerative disorders or post-traumatic stress syndrome, wherein said combination is formulated in a transdermal patch, characterized in that said transdermal patch comprises at least one transdermal nanofibrous film; in particular at least one transdermal nanofibrous electrospun film.

The present invention thus also relates to a transdermal patch comprising Tetrahydrocannabinol (THC) and Cannabidiol (CBD); wherein said patch comprises at least one transdermal nanofibrous film; in particular at least one transdermal nanofibrous electrospun film. Preferably said transdermal path is for use in the treatment of a disorder selected from the list comprising: pain, blisters, neurodegenerative disorders or post-traumatic stress syndrome.

In some embodiments, the present invention relates to a transdermal patch comprising THC.

In some embodiments, the present invention relates to a transdermal patch comprising CBD.

In some embodiments, the combination of CBD and THC may have a synergistic effect (e.g. in terms of analgesic response) on the treatment of at least one of the disorders.

In some embodiments, CBD may decrease at least one of the potential side-effects caused by THC and vice versa. CBD may for example antagonize side-effects such as tachycardia and sedation caused by THC.

In some embodiments, at least one of the cannabinoids of the combination may have an immunoregulatory effect or immunomodulatory effect or may otherwise influence the immune system in order to support the treatment of one of said disorders.

As used herein and unless otherwise specified, the term “cannabinoid” is to be understood as a compound effectuating an activity involving the endocannabinoid system.

As used herein and unless otherwise specified, the term “endocannabinoid system” is to be understood as a cell-signaling system composed of endocannabinoids, being for example endogenous ligands of cannabinoid receptors (CB₁ and CB₂), and cannabinoid receptor proteins being expressed at the height of the vertebrate central nervous and peripheral nervous system.

In some embodiments, the cannabinoids including THC and CBD may be synthetically produced and/or plant-derived.

In some embodiments, the cannabinoids may be derived from Cannabis plants belonging to the Cannabis sativa L. species. Subspecies thereof may include: Cannabis sativa ssp. Sativa and ssp. Indica.

In some embodiments, the combination may comprise at least one Cannabis plant metabolite, including in particular: cannabinoids, terpenes, terpenoids, triglycerides, sterols, alkanes, squalenes, tocopherols, alkaloids, flavonoids or carotenoids.

As used herein and unless otherwise specified, the term “cannabis plants” is to be understood as a genus of flowering plants in the family of the Cannabaceae. Three main species can be recognized: Cannabis sativa, Cannabis indica and Cannabis ruderalis.

In some embodiments, the combination comprising THC and CBD may comprise THC and CBD in a ratio of THC:CBD from about 1:1000, 1:500, 1:250 to about 1000:1, 500:1, 250:1, preferably from about 1:100, 1:50, 1:25 to about 100:1, 50:1, 25:1 and more preferably from about 1:10 to about 10:1.

In some embodiments, the combination comprising THC and CBD may comprise THC and CBD in a ratio of THC:CBD from about 2:1 to about 1:2

In some embodiments, the combination comprising THC and CBD may comprise THC and CBD in a ratio of THC:CBD from about 1:1 to about 1:1

In some embodiments, the combination comprising THC and CBD may comprise an amount of THC of about 0.0625, 0.125, 0.25, 0.50, 0.75; 1; 2; 3; 4; 5; 10; 20; 30; 40; 50; 60; 70; 80; 90; 100 mg THC up to about 200; 300; 400; 500; 600; 700; 800; 900; 1000 mg THC and preferably an amount of THC of about 0.0625 to 100 mg THC.

In some embodiments, the combination comprising THC and CBD may comprise an amount of CBD of about 0.0625, 0.125, 0.25, 0.50, 0.75, 1, 2, 3, 4, 5, 10, 20, 30, 40, 50, 60, 70, 80, 90, 100 mg CBD up to about 200, 300, 400, 500, 600, 700, 800, 900, 1000 mg CBD.

In some embodiments, the combination may comprise at least one of the list comprising: THC CBD or other cannabinoids.

In some embodiments, these “other cannabinoids” may be selected from the list comprising the following types of cannabinoids: delta-9-trans-tetrahydrocannabinol (Δ⁹-THC) type, delta trans-tetrahydrocannabinol (Δ⁸-THC) type, cannabigerol (CBG) type, cannabichromene (CBC) type, cannabidiol (CBD) type, cannabinodiol (CBND) type, cannabielsoin (CBE) type, cannabicyclol (CBL) type, cannabinol (CBN) type, cannabitriol (CBT) type or miscellaneaous-type cannabinoids.

The delta-9-trans-tetrahydrocannabinol (Δ⁹-THC) type includes molecules from the list comprising: Delta-9-tetrahydrocannabinol (THC), Delta-9-tetrahydrocannabinolic acid A (THCA-A), Delta-9-tetrahydrocannabinolic acid B (THCA-B), Delta-9-tetrahydrocannabivarin (THCV), Delta-9-tetrahydrocannabivarinic acid (THCVA), Delta-9-tetrahydrocannabiorcol (THCO), Delta-9-trans-tetrahydrocannabiorcolic acid (THCOA), Delta-9-tetrahydrocannabinol-C4 (THC-C4), Delta-9-trans-tetrahydrocannabinolic acid-C4 (THCA-C4), β-fenchyl-Delta-9-tetrahydrocannabinolate, α-fenchyl-Delta-9-tetrahydrocannabinolate, epi-bornyl-Delta-9-tetrahydrocan nabinolate, bornyl-Delta-9-tetrahydrocannabinolate, α-terpenyl-Delta-9-tetrahydrocannabinolate, 4-terpenyl-Delta-9-tetrahydrocannabinolate, α-cadinyl-Delta-9-tetrahydrocannabinolate, γ-eudesmyl-Delta-9-tetrahydrocannabinolate or Cannabisol.

The delta-8-trans-tetrahydrocannabinol (Δ⁸-THC) type includes molecules from the list comprising: Delta-8-trans-tetrahydrocannabinol (Δ8-THC) or Delta-8-trans-tetrahydrocannabinolic acid (Δ8-THCA).

The cannabigerol (CBG) type includes molecules from the list comprising: Cannabigerol (CBG), Cannabigerolic acid (CBGA), Cannabigerol monomethyl ether (CBGM), Cannabigerolic acid monomethyl ether (CBGAM), Cannabigevarin (CBGV), Cannabigerovarinic acid (CBGVA), Cannabinerolic acid ((Z)-CBGA), γ-eudesmyl-Cannabigerolate, α-cadinyl-Can nabigerolate, 5-acetyl-4-hydroxycannabigerol, 4-acetoxy-2-geranyl-5-hydroxy-3-n-pentylphenol, (±)-6,7-trans-epoxycannabigerolic acid, (±)-6,7-cis-epoxycannabigerolic acid, (±)-6,7-cis-epoxycannabigerol, (±)-6,7-trans-epoxycannabigerol, Carmagerol-dihydroxy-CBG or Sesquicannabigerol.

The cannabichromene (CBC) type includes molecules from the list comprising: Cannabichromene (CBC), Cannabichromenic acid (CBCA), Cannabichromevarin (CBCV), Cannabichromevarinic acid (CBCVA), Cannabichromene C3 (CBC-C3), (±)-4-acetoxycannabichromene, (±)-3″-hydroxy-Δ⁴″-cannabichromene or (−)-7 hydroxycannabichromane.

The cannabidiol (CBD) type includes molecules from the list comprising: Cannabidiol (CBD), Cannabidiolic acid (CBDA), Cannabidivarin (CBDV), Cannabidivarinic acid (CBDVA), Cannabidiol monomethyl ether (CBDM), Cannabidiorcol (CBD-C1), Cannabidiol-C4 (CBD-C4) or Cannabimovone.

The cannabinodiol (CBND) type includes molecules from the list comprising: Cannabinodiol (CBND-C5) or Cannabinodivarin (CBND-C3).

The cannabielsoin (CBE) type includes molecules from the list comprising: Cannabielsoin (CBE), Cannabielsoic acid A (CBEA-A), Cannabielsoic acid B (CBEA-B), Cannabielsoin-C3 (CBE-C3), Cannabielsoic-C3 acid B (CBEA-C3 B), Cannabiglendol-C3-OH-iso-HHCV-C3, Dehydrocannabifuran (DCBF) or Cannabifuran (CBF).

The cannabicyclol (CBL) type includes molecules from the list comprising: Cannabicyclol (CBL), Cannabicyclolic acid (CBLA) or Cannabicyclovarin-CBLV (CBL-C3).

The cannabinol (CBN) type includes molecules from the list comprising: Cannabinol (CBN), Cannabinolic acid (CBNA), Cannabivarin-CBV (CBN-C3), Cannabinol-C4 (CBN-C4), Cannabinol-C2 (CBN-C2), Cannabiorcol (CBN-C1), Cannabinol methyl ether (CBNM), 4-terpenyl-Cannabinolate, 8-Hydroxycannabidiol (8-OH-CBN) or 8-Hydroxycannabidiolic acid (8-OH-CBNA).

The cannabitriol (CBT) type includes molecules from the list comprising: (−)-trans-Cannabitriol ((−)-trans-CBT-05), (+)-trans-Cannabitriol ((+)-trans-CBT-05), cis-Cannabitriol ((±)-CBT-05), (−)-trans-10-ethoxy-9-hydroxy-Δ6a(10a)-tetrahydrocannabinol ((−)-trans-CBT-OEt-05), trans-Cannabitriol-C3 ((±)-trans-CBT-C3), CBT-C3-homoloog, trans-10-ethoxy-9-hydroxy-Δ6a(10a)-tetrahydrocannabivarin-C3 ((−)-trans-CBT-OEt-C3), 8,9-dihydroxy-Δ6a(10a)-tetrahydrocannabinol (8,9-Di-OH-CBT-05), Cannabidiolic acid A cannabitriol ester (CBDA-C5 9-OH-CBT-05 ester), Cannabitriolvarin (CBTV) or ethoxy-Cannabitriolvarin (CBTVE).

The miscellaneaous-type cannabinoids includes molecules from the list comprising: Cannabifuran (CBF), Dehydrocannabifuran (DCBF), Cannabitetrol (CBTT), Cannabiripsol (CBR), Cannabicitran (CBR-C3), Cannabioxepane (CBX), Cannabicoumaronone (CBCON), Cannabicoumaronic acid, Cannabiglendol-C3 (OH-iso-HHCV-C3), 10-Oxo-Δ6a(10a)-tetrahydrocannabinol (OTHC), (−)-Δ0-cis-(6aS,10aR)-tetrahydrocannabinol (cis-Δ9-THC), 4-acetoxy-2-geranyl-5-hydroxy-3-n-pentylphenol, 2-geranyl-5-hydroxy-3-n-pentyl-1,4-benzoquinone, 5-acetoxy-6-geranyl-3-n-pentyl-1,4-benzoquinone, 8α-hydroxy-Δ9-tetrahydrocannabinol, 8β-hydroxy-Δ9-tetrahydrocannabinol, 10α-hydroxy-Δ8-tetrahydrocannabinol, 10β-hydroxy-Δ8-tetrahydrocannabinol, 10α-hydroxy-Δ9,11-hexahydrocannabinol, 9β,10β-epoxyhexahydrocannabinol or 11-acetoxy-Δ9-tetrahydrocannabinolic acid A.

As used herein and unless otherwise specified, the term “transdermal patch” is to be understood as an adhesive patch for placement upon the skin in order to deliver a specific dose of at least one active pharmaceutical ingredient (API) through the skin into the bloodstream. These types of pharmaceutical formulations are able to effectuate e.g. a controlled release of the API, minimizing the risk of burst plasma concentrations of the API(s) in the body throughout the API delivery. Generally, the main components of transdermal patches may be selected from the list comprising: a liner, an API, an adhesive, a membrane/film, a backing layer and a permeation enhancer. Generally, the liner will be removed prior to use, the API will accommodate the therapeutic effect, the adhesive serves to adhere the patch components to the skin, the membrane or film aids in the control of drug release, the backing layer protects the patch from the outer environment and the permeation enhancer increase the transdermal delivery of the API.

As used herein and unless otherwise specified, the term “nanofibrous electrospun film” is to be understood as a network of nanofibers which are produced using an electrohydrodynamic process, wherein these nanofibers have diameters of some microns to few ten nanometers. These nanofibers are often created using various polymers and can be loaded with active pharmaceutical ingredients (e.g. nano-encapsulated drug nanoparticles). These nanofibers are processed into films or membranes having specific properties such as e.g. high surface to volume ratios, flexible surface functional groups and excellent mechanical performance. The loading of these films with API's may be realized using different methods such as e.g. blending, coaxial electrospinning, emulsion electrospinning, physical adsorption and chemical immobilization.

As used herein and unless otherwise provided, an electrohydrodynamic processes is to be understood as a process which uses an electrically charged jet of polymer solution for the fabrication of micro- or nanofibers (electrospinning) or micro- or nanoparticles (electrospraying). The main apparatus used for both of these processes is almost the same. Both need electric voltage to induce charge on the droplet, which at optimized electric field leads to micro- or nanofibers and micro- or nanoparticles.

As used herein and unless otherwise provided, the term “electrospinning” is to be understood as a fibre production method wherein electric force is applied in order to draw charged threads based on polymer solutions or polymer melts and having diameters from some microns to few ten nanometers. In electrospinning, the polymer solution in the capillary is induced with free charges by a high voltage potential.

As used herein and unless otherwise provided, the term “electrospraying” is to be understood as a particle production method wherein an electrical field is used allowing for the breakdown of a conductive liquid jet, flowing through a capillary nozzle, into fine droplets with high monodispersity. Electrospraying is a versatile and inexpensive way to produce nanoparticles and nanosuspensions.

The difference between electrospinning and electrospraying techniques is based on the concentration of the polymer solution. When the solution concentration is high, the jet from the hemispherical surface of the droplet (Taylor cone) is stabilized, and elongation takes place by whipping instability mechanism. If the solution concentration is low, the jet is destabilized due to varicose instability and hence fine droplets are formed. These highly charged droplets are self-dispersing in space, thereby preventing droplet agglomeration and coagulation.

Further, the size of the final product can be controlled by manipulating the governing factors such as the system, solution, instrumental and ambient parameters. The system parameters include the molecular weight and the microstructural feature of the polymer. The type and concentration of the polymer and solvent used, determine the solution properties namely pH, conductivity, viscosity and surface tension. The instrumental parameters include electrical potential applied, flow rate of the solution, distance between the tip of the needle and the collector and occasionally the nature of collector material. Additionally, the ambient conditions such as the temperature, humidity and air velocity in the process chamber together determine the rate of evaporation of the solvent from the electrospun or electrosprayed product. Thus products at micron (>1 mm), submicron or nano (10-1000 nm) scale can be obtained.

In some embodiments, said transdermal nanofibrous film is generated by an electrohydrodynamic processes. In another embodiment, said transdermal nanofibrous film is generated by electrospinning.

In the context of the present invention, said transdermal film may be generated by electrospinning as well as electrospraying, or a combination thereof.

In another embodiment, said transdermal nanofibrous film is generated by incorporating electrosprayed nano-encapsulated drug nanoparticles (dry powder) into a transdermal nanofibrous film via electrospinning as described in example 2.

In yet another embodiment, a transdermal granular film may be generated by electrospraying.

In a specific embodiment, said transdermal patch may comprise at least one transdermal granular electrosprayed film.

For example, depositing dry nano-encapsulated THC and/or CBD particles made by electrospray onto a solid substance forms a granular film.

For the sake of clarity, the components, concentrations, ratio's, . . . defined herein below for “nanofibrous film” may equally apply for a “granular film”.

In some embodiments, the drug release of the transdermal nanofibrous film may be altered by tuning the porosity of the nanofibrous film.

The porosity may facilitate the swelling of the nanofibrous film and the diffusion of the active substances from the nanofibrous film through the skin.

In some embodiments, the drug release of the transdermal nanofibrous film may be altered by tuning the wettability of the nanofibrous film (e.g. by choosing certain types of polymers).

The use of a transdermal patch may increase the bioavailability of at least one of the active substances compared to other formulations. For example, when using oral drug delivery, the drugs are subject to hepatic drug metabolization (hereinafter also called: first pass effect) before reaching the systemic circulation. Cannabinoids in particular are substantially deactivated when subject to this first pass effect. The use of formulations avoiding this first pass effect, such as transdermal patches, therefore increases the overall bioavailability of cannabinoids, making it a suitable pharmaceutical formulation.

Transdermal patches accommodate a transdermal uptake of the active substances once applied topically. Besides the avoidance of first-pass metabolism and the production of psycho-active metabolites (11-OH-THC), the use of transdermal patches may enable a relatively fast onset of action and for example a prolonged release and/or sustained release of certain active substances,

For the avoidance of doubt, the components, concentrations, ratio's, . . . defined herein below for “a combination” may equally apply for a “transdermal patch”, “nanofibrous film” as well as for the starting material (or liquid feed) to produce such formulation.

As used herein, the term “liquid feed” is to be understood as starting material or starting liquid to be converted to a nanofibrous film or transdermal patch comprising an active pharmaceutical ingredient (API) such as CBD, THC or CBD/THC and optionally one or more other excipients and/or carriers.

In addition, the concentration of a component in the liquid feed may not necessarily be the same as the final concentration of the transdermal patch of the present invention.

As used herein and unless otherwise specified, the term “excipient” is to be understood as a substance formulated alongside the API, which may be used for long-term stabilization, bulking up formulations that contain potent active ingredients in small amounts (thus often also referred to as “bulking agents”, “fillers”, or “diluents”), or to confer a therapeutic enhancement on the active ingredient in the final dosage form, such as facilitating drug absorption, reducing viscosity or enhancing solubility. Excipients may also be used in the manufacturing process, to aid in the handling of the active substance concerns.

In some embodiments, the transdermal patch and/or film may comprise THC in a concentration of at least about 0.5, 1, 2, 3, 4, 5, 6, 7, 8, 9, 10, 11, 12, 13, 14, 15, 16, 17, 18, 19, 20%.

In some embodiments, the transdermal patch and/or film may comprise CBD in a concentration of at least about 0.5, 1, 2, 3, 4, 5, 6, 7, 8, 9, 10, 11, 12, 13, 14, 15, 16, 17, 18, 19, 20%.

In some embodiments, the transdermal patch and/or film may comprise a mixture of THC/CBD in a concentration of at least about 0.5, 1, 2, 3, 4, 5, 6, 7, 8, 9, 10, 11, 12, 13, 14, 15, 16, 17, 18, 19, 20%.

In some embodiments, the transdermal patch and/or film may comprise at least one preservative excipient, more in particularly a carrier oil, such as selected from the list comprising: medium-chain triglyceride (MCT) oil, hemp seed oil, olive oil, avocado oil, sunflower oil, safflower oil, palm oil, flaxseed oil, peanut oil, argan oil, or sesame oil.

In some embodiments, the combination comprising a cannabinoid and carrier oil may be in a ratio of cannabinoid:carrier from about 1:1000, 1:500, 1:250 to about 1000:1, 500:1, 250:1, preferably from about 1:100, 1:50, 1:25 to about 100:1, 50:1, 25:1 and more preferably from about 1:10 to about 10:1.

In some embodiments, the transdermal patch and/or film may comprise at least one polymer, more in particularly selected from the list comprising:
(a) natural polymers (e.g. sodium alginate, pectin, tragacanth, gelatin, carrageenan, agarose)
(b) synthetic polymers (e.g. copolymers of methyl vinyl ether and methacryclic acid, polymethacrylate polyvinyl alcohols, polyamides, polycarbonates, polyalkylene glycols, polyvinyl ethers, polymethacrylic acid, Polymethyl methacrylic acid, methylcellulose, ethylcellulose, hydroxypropyl cellulose, hydroxypropyl methylcellulose, sodium carboxymethyl cellulose), Carbopol, polycarbophil, polyethylene glycol (PEG), polypropyleneglycol (PPG), propylene glycol (PG),
(c) biocompatible polymers (esters of hyaluronic acid, polyvinyl acetate, ethylene glycol),
(d) biodegradable polymers (e.g. polylactides, polyglycolides, polylactide co-glycolides, polycaprolactones, polyalkyl cyanoacrylates, polyisobutylcyanoacrylate or thiolated polymers, polyorthoesters, polyphosphoesters, polyanhydrides, polyphosphazenes, chitosan, polyethylene oxide and
(e) other polymers including: glycerol, sorbitol, mannitol, soluble starch, xylitol, glucose syrups or polydextrose.

In some embodiments, the transdermal patch and/or film may comprise at least one polymer at a concentration of at least about 0.5, 1, 2, 3, 4, 5, 6, 7, 8, 9, 10, 11, 12, 13, 14, 15, 16, 17, 18, 19, 20%.

In a specific embodiment, said transdermal patch and/or film comprises least one polymer, preferably polyethylene oxide.

In another embodiment, said transdermal patch and/or film comprises 8% polyethylene oxide.

In a specific embodiment, said transdermal patch and/or film comprises polycaprolactone.

In a specific embodiment, said transdermal patch and/or film comprises 8% polycaprolactone.

In some embodiments, the transdermal patch and/or film may comprise a solvent such as selected from the list comprising: inorganic solvent and organic solvent.

The solvents include molecules from the list comprising: ethanol, propanol, propylene glycol, glycerol, polyethylene glycol, methanol, chloroform, and sub-micron liposomal dispersion (microemulsions and micellar solutions). In some embodiments, at least a part of the cannabinoid(s) within the transdermal patch may be dissolved within said solvent.

In a specific embodiments, at least a part of the cannabinoid(s) within the transdermal patch and/or film may be dissolved within a mixture of chloroform:methanol.

In some embodiments, said mixture of chloroform and methanol to dissolve cannabinoid(s) within the transdermal patch and/or film may be in a ratio chloroform:methanol of about 10:90, 20:80, 30:70, 40:60, 50:50 to about 60:40, 70:30, 80:20, 90:10.

In some embodiments, the combination may be administered multiple times a week and more specifically at least once, twice, three times, four times, five times, six times, seven times, eight times, nine times, ten times a day.

In another embodiment, the combination may be administered not more than three, four, five, six, seven, eight, nine, ten times a week.

In some embodiments, the combination may be administered on a daily basis, more specifically at least once or twice a day.

In some embodiments, the combination may be administered by the subject. Transdermal patches are generally suitable to be administered by the subject without any additional assistance. However, if the condition of the subject does not permit self-administration, the transdermal patches may be administered by others (e.g. nurses).

In some embodiments, the transdermal patch and/or film may comprise a minimum amount of THC of at least about 1, 2, 3, 4, 5, 10, 15, 20, 25, 30, 40, 50 wt %.

In some embodiments, the transdermal patch and/or film may comprise a minimum amount of CBD of at least about 1, 2, 3, 4, 5, 10, 15, 20, 25, 30, 40, 50 wt %.

In some embodiments, the transdermal patch and/or film may comprise a minimum amount of THC of up to about 60, 70, 80, 90, 95 wt %.

In some embodiments, the transdermal patch and/or film may comprise a minimum amount of CBD of up to about 60, 70, 80, 90, 95 wt %.

As used herein and unless otherwise specified, the term “wt %” is also referred to as weight percentage and is to be understood as the mass of a component divided by the total mass of the mixture and then multiplied by 100.

In a following embodiment, said nanofibrous film comprises nano-encapsulated THC and/or CBD.

As used herein and unless otherwise provided, nano-encapsulation is to be understood as a method of encapsulating therapeutic agents in a secondary material (may also be referred to as: outer shell or matrix) in order to improve certain aspects of these therapeutic agents, such as their bioavailability, hydrophilic properties, drug release properties, systemic toxicity, cellular response and cellular uptake. Drug nano-encapsulation may be achieved using among others electrospraying techniques. In a particular embodiment, a transdermal granular film may be formed by collecting dry nano-encapsulation particles made by electrospray onto a solid substance.

In some embodiments, the nano-encapsulation of cannabinoids (e.g. THC, CBD) provides for improved properties of the cannabinoids, more specifically improved hydrophilic properties, which may result in enhanced plasma concentrations of THC when applied within a transdermal patch.

In other embodiments, the nano-encapsulation of cannabinoids reduces the risk of excessive dosing and/or burst-release due to an improved ability of controlled drug release properties within the transdermal patch.

In a next embodiment, said transdermal patch is selected from the list comprising: reservoir patch, matrix patch, vapor patch or drug-in-adhesive patch. Said drug-in-adhesive patch may selected from the list comprising: single-layer drug-in-adhesive patch or multi-layer drug-in-adhesive patch.

As used herein and unless otherwise provided, a reservoir patch is to be understood as a transdermal patch having a separate drug layer, wherein the drug is enclosed in a rate-controlling membrane and wherein the drug is released according to a zero order release rate.

As used herein and unless otherwise provided, a matrix patch (may also be referred to as: monolithic patch) is to be understood as a transdermal patch having a drug layer of a semisolid matrix comprising a drug solution or a drug suspension. Furthermore, the drug layer is surrounded by an adhesive layer. Generally, the skin characteristics will mainly determine the rate of drug diffusion into the bloodstream.

As used herein and unless otherwise provided, a vapor patch is to be understood as a transdermal patch having an adhesive layer which has multiple functions. The first being holding the various layers of the transdermal patch together and the second being the release of vapor, such as e.g. essential oils.

As used herein and unless otherwise provided, a single-layer drug-in-adhesive patch is to be understood as a transdermal patch wherein the drug is incorporated directly into the adhesive rather than in a separate layer. In this case the adhesive layer has multiple functions, the first being holding the various layers of the transdermal patch together and the second being the release of the drug substances,

The multi-layer drug-in-adhesive patch is similar to the single-layer variant, but adds another layer of drug-in-adhesive which may or may not be separated by a membrane. Generally, the first drug-in-adhesive layer may effectuate immediate release of drug substances wherein the second drug-in-adhesive layer may effectuate controller release of the drug substances from a reservoir. The drug release mainly depends on membrane permeability as well as the diffusion of drug molecules.

It is important to note that, depending on the subtype of transdermal patches, certain aspects of the formulations may vary, such as the drug release pattern and/or the bioavailability.

As used herein and unless otherwise specified, the term “drug release pattern” is to be understood as the release rate of the active drug substance immediately after administration. The selected pharmaceutical formulation in which the drug is formulated mainly determines the drug release pattern.

As used herein and unless otherwise specified, the term “extended-release” is to be understood as a modified-release dosage form which slowly releases the active substance(s), ultimately allowing for a reduced dosage frequency.

Examples include: sustained-release and controlled-release drug products.

As used herein and unless otherwise specified, the term “delayed-release” is to be understood as a modified-release dosage form which is often coated in order to delay the release of the active substance(s) until these have passed the stomach, preventing inactivation of the active substance(s) and/or irritation of the gastric mucosa. Examples include: enteric-coated drug products.

As used herein and unless otherwise specified, the term “targeted-release” is to be understood as a modified-release dosage form which releases the drug(s) at least in close proximity of the intended physiologic site of action.

In a next embodiment, the present invention also provides a combination comprising Tetrahydrocannabinol (THC) and Cannabidiol (CBD) for use in the treatment of a disorder selected from the list comprising: pain, blisters, neurodegenerative disorders or post-traumatic stress syndrome; wherein said combination is a transdermal patch and at least one of said THC and/or CBD is a prodrug. Said transdermal patch may be further characterized in that it comprises at least one transdermal nanofibrous film, comprising said prodrugs.

As used herein and unless otherwise specified, the term “prodrug” is to be understood as a pharmacologically inactive compound that is metabolized into an active form after uptake within the body. Generally, the inactive prodrug is used to improve certain pharmacokinetic characteristics of the drug substance, involving the absorption, distribution, metabolization and/or excretion of the drug concerned.

In some embodiments, the prodrug form of THC and/or CBD improves the overall bioavailability of the drug substance(s).

In some embodiments, at least one of the layers of the transdermal patch comprises a cannabinoid prodrug.

In some embodiments, the prodrug form of THC and/or CBD reduces the adverse or unintended effects of the drug substance(s).

In some embodiments, said skin penetration enhancer (PE) (may also be referred to as a skin permeation enhancer or an absorption modifying excipient (AME)) may be added to improve transdermal absorption.

In some embodiments, said transdermal patch and/or film may further comprise a skin penetration enhancer such as selected from the list comprising: isopropyl myristate, decyl oleate, oleyl alcohol, octyldodecanol, propylene glycol, triacetin, cocoyl caprylocaprat; alcohols such as short chain alcohols (Ethanol, Isopropyl alcohol); Long chain alcohols (Decanol, Hexanol, Lauryl alcohol, Myristyl alcohol, Octanol, Octyl dodecanol, Oleyl alcohol), amides such as Cyclic amides (azone), esters such as alkyl esters (Ethyl acetate); Benzoic acid esters (Octyl salicylate, Padimate 0); Fatty acid esters (Ethyl oleate, Glyceryl monoleate, Glyceryl monocaprate, Glyceryl tricaprylate Isopropyl myristate, Isopropyl palmitate, Propylene glycol monolaurate, Propylene glycol monocaprylate), Ether alcohols (Transcutol®), Fatty acids (Lauric acid, Linoleic acid, Linolenic acid, Myristic acid, Oleic acid, Palmitic acid, Stearic acid, Isostearic acid), Glycols (Dipropylene glycol, Propylene glycol, 1,2-butylene glycol, 1,3-butylene glycol), Pyrrolidones (N-methyl-2-pyrrolidone, 2-pyrrolidone), Sulphoxides (Decylmethyl sulphoxide, Dimethyl sulphoxide), Surfactants such as sorbitan esters (SPANS), Anionic surfactants (Sodium lauryl sulphate); Cationic surfactants (Alkyl dimethylbenzyl ammonium halides, Alkyl trimethyl ammonium halides Alkyl pyridinium halides); Non-ionic surfactants (Brij 36T, Tween 80), Terpenes such as Monoterpenes (Eugenol, d-Limonene, Menthol, Menthone) and Sesquiterpenes (Farnesol, Neridol).

In some embodiments, said transdermal patch and/or film comprises a skin penetration enhancer at a concentration of at least about 0.0625, 0.25, 0.5, 0.75 1, 2, 3, 4, 5, 6, 7, 8, 9, 10, 11, 12, 13, 14, 15, 16, 17, 18, 19, 20% (w/w)

In another embodiment, said transdermal patch and/or film comprises SPAN.

In another embodiment, said transdermal patch and/or film comprises oleic acid.

In a specific embodiment, said transdermal patch and/or film comprises 1% SPAN.

In another specific embodiment, said transdermal patch and/or film comprises 1% oleic acid. In some embodiments, said transdermal patch and/or film may further comprise additives, in particular antioxidants, more in particular vitamins such as vitamin C (ascorbic acid), vitamin E (Tocophersolan; TGPS); uric acid, lipoic acid, glutathione.

In some embodiments, said transdermal patch and/or film comprises an antioxidant at a concentration of at least about 0.0625, 0.25, 0.5, 0.75 1, 2, 3, 4, 5, 6, 7, 8, 9, 10, 11, 12, 13, 14, 15, 16, 17, 18, 19, 20% (w/w).

In another embodiment, said transdermal patch and/or film may comprise ascorbic acid.

In another embodiment, said transdermal patch and/or film may comprise TGPS.

In a specific embodiment, said transdermal patch may comprise 1% ascorbic acid.

In another specific embodiment, said transdermal patch may comprise 1% TGPS.

In another specific embodiment, said transdermal patch and/or film may comprise 9% THC, 8% polyethylene oxide, 1% SPAN, 1% ascorbic acid.

In another specific embodiment, said transdermal patch and/or film may comprise 4% THC, 8% polyethylene oxide, 1% SPAN, 1% ascorbic acid.

In another specific embodiment, said transdermal patch and/or film may comprise 9% THC, 8% polyethylene oxide, 1% oleic acid, 1% ascorbic acid.

In another specific embodiment, said transdermal patch and/or film may comprise 9% THC, 8% polyethylene oxide, 1% oleic acid, 1% TGPS.

In another specific embodiment, said transdermal patch and/or film may comprise 4% THC, 8% polyethylene oxide, 1% oleic acid, 1% TGPS.

In another specific embodiment, said transdermal patch and/or film may comprise 9% THC, 8% polyethylene oxide, 1% SPAN, 1% TGPS.

In another specific embodiment, said transdermal patch and/or film may comprise 4% THC, 8% polyethylene oxide, 1% SPAN, 1% TGPS.

In a next embodiment, the present invention discloses a combination as defined herein, for use in the treatment of a disorder selected from the list comprising: pain, blisters, neurodegenerative disorders or post-traumatic stress syndrome, wherein said skin blisters are caused by burns or other related traumas or a disorder selected from the list comprising: skin allergies, different forms of eczema, bullous pemphigoid, bullous impetigo, dermatitis herpetiformis, pemphigus vulgaris, mucous membrane pemphigoid, pemphigoid gestationis, epidermolysis bullosa, pemphigus foliaceous or toxic epidermal necrolysis.

As used herein and unless otherwise specified, the term “blisters” is to be understood as a bulge of the upper layers of the skin, filled with body fluid such as serum or plasma and generally caused by for example infections, burning or friction, or other related traumas or a disorder selected from the list comprising: skin allergies, different forms of eczema, bullous pemphigoid, bullous impetigo, dermatitis herpetiformis, pemphigus vulgaris, mucous membrane pemphigoid, pemphigoid gestationis, epidermolysis bullosa, pemphigus foliaceous or toxic epidermal necrolysis.

As used herein and unless otherwise specified, the term “skin allergies” is to be understood as a reaction to an allergen or irritant, which may lead to symptoms such as itchiness, redness of the skin, rashes and blistering of the skin. Treatment includes treatment of the underlying conditions which cause the skin allergy and treatment of the symptoms.

As used herein and unless otherwise specified, the term “eczema” (may also be referred to as: dermatitis”) is to be understood as a group of diseases being only partly interrelated and resulting in inflammation of the skin and characterized by itchiness, red skin, rashes, skin thickening and small blisters.

In some embodiments, the different forms of eczema may be selected from the list comprising: atopic dermatitis, contact dermatitis, dyshidrotic eczema, nummular eczema, seborrheic dermatitis or stasis dermatitis.

As used herein and unless otherwise specified, the term “bullous pemphigoid” is to be understood as an autoimmune pruritic skin disease. It is a type of pemphigoid, which is a group of autoimmune blistering skin diseases. Bullous pemphigoid may by characterized by the formation of blisters within the (epi)dermal skin layers and the formation of anti-hemidesmosome antibodies.

As used herein and unless otherwise specified, the term “bullous impetigo” is to be understood as a bacterial skin infection resulting in large blisters, mainly in skin fold areas (e.g. groin, armpit). The blisters are caused by exfoliative toxins which are produced by Staphylococcus aureaus, causing the intercellular connections of the epidermis to fall apart.

As used herein and unless otherwise specified, the term “dermatitis herpetiformis” is to be understood as a chronic autoimmune skin condition characterized by fluid-filled blisters, chronic papulovesicular eruptions and intense itchiness. Dermatitis herpetiformis is a cutaneous manifestation of Coeliac disease and symptoms are chronic and are linked to the amount of gluten ingested.

As used herein and unless otherwise specified, the term “pemphigus vulgaris” is to be understood as a chronic skin disease characterized by blistering of the skin. It is a type II hypersensitivy reaction with antibody formation against desmosomes. The attack of these desmosomes by the antibodies causes the skin layers to be separated, which resembles blisters.

As used herein and unless otherwise specified, the term “mucous membrane pemphigoid” (may also be referred to as: MMP″) is to be understood as a group of chronic autoimmune subepithelial blistering diseases which primarily involves the mucous membranes and sometimes the skin.

As used herein and unless otherwise specified, the term “pemphigoid gestationis” is to be understood as a pregnancy-associated autoimmune skin disease. Often, an itchy rash develops into blisters. It may sometimes also be referred to as herpes gestationis.

As used herein and unless otherwise specified, the term “epidermolysis bullosa” is to be understood as a group of genetic skin conditions characterized in the formation of blisters of the mucous membranes and the skin. The conditions cannot be cured, although wound care and pain relief are often applied.

As used herein and unless otherwise specified, the term “pemphigus foliaceous” is to be understood as an autoimmune blistering disease of the skin. Skin lesions are often crusted erosions with an erythematous base.

As used herein and unless otherwise specified, the term “toxic epidermal necrolysis” is to be understood as a potentially life-threatening skin disorder, resulting in skin blistering and affected mucous membranes.

As used herein and unless otherwise specified, the term “neurodegenerative disorders” is to be understood as an umbrella term for a number of conditions primarily affecting brain neurons. These conditions are often incurable and debilitating and often result in progressive degeneration and/or death of nerve cells.

As used herein and unless otherwise specified, the term “post-traumatic stress syndrome (also referred to as: PTSS)” is to be understood as a mental health condition often triggered by the experience or witnessing of a terrifying event. PTSS symptoms include intrusive memories, negative changes of thoughts and mood, changes relating to physical and emotions reactions and avoidance.

In a further embodiment, said neurodegenerative disorder is selected from the list comprising: Alzheimer's disease (AD) and other dementias, Parkinson's disease (PD) and PD-related disorders, Essential Tremor, Multiple System Atrophy, Huntington's disease or Motor Neuron Diseases (MND).

As used herein and unless otherwise specified, the term “Alzheimer's disease (AD)” is to be understood as a chronic neurodegenerative disease. The disease is often characterized by among others: disorientations, mood swings and behavioral issues. Besides that, it is the most common cause of dementia.

As used herein and unless otherwise specified, the term “Parkinson's disease (PD)” is to be understood as a neurodegenerative disease of the central nervous system, affecting the motor system. Symptoms include rigidity, walking difficulties and shaking.

As used herein and unless otherwise specified, the term “PD-related disorders” is to be understood as a group of disorders related to PD itself and/or the pharmacological management of the disease, including dopamine deficiency syndrome, dopamine dependency syndrome, impulse control disorders and dopamine dysregulation syndrome.

As used herein and unless otherwise specified, the term “Essential Tremor” is to be understood as a progressive neurological disorder, which may affect all parts of the body. It causes involuntary, rhythmic contractions and relaxations of certain muscle groups. Furthermore, it is the most common movement disorder.

As used herein and unless otherwise specified, the term “Multiple System Atrophy” is to be understood as a progressive neurodegenerative disorder, caused by progressive degeneration of neurons in several parts of the brain (e.g. cerebellum and basal ganglia). It is characterized by among others slow movement, tremors and autonomic dysfunction.

As used herein and unless otherwise specified, the term “Huntington's disease” is to be understood as an inherited disease causing progressive degeneration of brain nerve cells, resulting in cognitive, physical and psychiatric disorders. Signs and symptoms tend to develop between the age of 30 to 40.

As used herein and unless otherwise specified, the term “Motor Neuron Diseases (MND)” is to be understood as a group of neurodegenerative diseases affecting motor neurons, causing moement-related symptoms (e.g. muscle weakness). The group includes: progressive bulbar palsy, amyotrophic lateral sclerosis, progressive muscular atrophy, monomelic amyotrophy and primary lateral sclerosis.

In a next embodiment, the present invention provides a combination as defined herein for use in the treatment of a disorder selected from the list comprising: pain blisters, neurodegenerative disorders or post-traumatic stress syndrome is disclosed, wherein said use includes the alleviation of secondary symptoms caused by said disorders.

In some embodiments, a long-term treatment may be necessary for the alleviation of said secondary symptoms. Some of the disorders which can be treated with the combination, or at least one of the cannabinoids of the combination require a long-term treatment in order to sufficiently alleviate the secondary symptoms associated with said disorders.

In some embodiments, this might involve a lifelong treatment. In other embodiments, this may be a treatment up until complete disappearance of the secondary symptoms caused by the disorders.

In some embodiments, said secondary symptoms may be chronic or recurring symptoms.

In a further embodiment, the present invention provides a combination as defined herein for use in the treatment of a disorder selected from the list comprising: pain, blisters, neurodegenerative disorders or post-traumatic stress syndrome is disclosed, wherein said use includes the alleviation of secondary symptoms caused by said disorders such as selected from the list comprising secondary pain, itch, secondary impetigos, swelling, inflammation or bacterial infection.

For the sake of clarity, when referring to pain, it can be understood as primary pain or secondary pain. In the context of the present invention, the term “primary pain” or just “pain” is to be understood as pain that is associated with significant emotional distress or functional disability in which no underlying condition adequately accounts for the pain or its impact. Examples of primary pain conditions include fibromyalgia, complex regional pain syndrome, headache, migraine, irritable bowel syndrome, or non-specific low-back pain.

In the context of the present invention, the term “secondary pain” is to be understood as pain that may initially be regarded as a symptom of other diseases having said disease being the underlying cause. Examples of secondary pain are pain related to cancer, pain related to blisters, surgery, injury, internal disease, disease in the muscles, bones or joints, headaches or nerve damage. Primary pain and secondary pain can coexist.

In a further embodiment, said primary pain is selected from the list comprising: headaches, migraine, physiological pain or, physical ailments.

As used herein, and unless otherwise specified, the term “headache” is to be understood as the symptom of pain in the face, head, or neck. It can occur as a tension-type headache (normal headache that cause pain in the head, face, or neck), cluster headache, sinus headache, or migraine. Cluster headaches are severely painful headaches that occur on one side of the head and come in clusters i.e. cycles of headache attacks, followed by headache-free periods. Sinus headaches co-occur with sinus infection symptoms like fever, stuffy nose, cough, congestion, and facial pressure. Migraine is a headache that is intense and severe and often have other symptoms in addition to head pain such as nausea, pain behind one eye or ear, pain in the temples, seeing spots or flashing lights, sensitivity to light and/or sound, temporary vision loss, vomiting.

As used herein and unless otherwise specified, the term “anti-inflammatory property” is to be understood as a property appointed to for example substances/compounds/treatment/drugs which reduces inflammation or swelling.

As used herein and unless otherwise specified, the term “analgesic property” is to be understood as a property appointed to for example substances/compounds/treatment/drugs which reduce pain, for example by interacting in various ways on peripheral and central nervous systems.

As used herein and unless otherwise specified, the term “antipruritic property” is to be understood as a property appointed to for example substances/compounds/treatment/drugs which inhibit itch (also referred to as: pruritis).

As used herein and unless otherwise specified, the term “antifungal properties” is to be understood as a property appointed to for example substances/compounds/treatment/drugs which prevent or treat various fungal conditions.

As used herein and unless otherwise specified, the term “antibacterial properties” is to be understood as a property appointed to for example substances/compounds/treatment/drugs which suppresses bacterial growth or their ability to reproduce or complete eliminates bacteria.

The analgesic effects of THC are linked to the agonism of cannabinoid receptors CB₁ and CB₂. Other effects comprise muscle relaxation and antiemesis. CBD, on the other hand shows activity including: 5-HT_{1A} receptor agonism, GPR55 antagonism, negative allosteric modulation of CB₁, TRPV1 activation, PPARγ activation and reuptake inhibition. CBD also appears to show activity at both CB₁ and CB₂ receptors while indirectly activating the endogenous cannabinoid signalling. All of this is believed to effectuate the anxiolytic, neuroprotective, anti-inflammatory and immunomodulatory properties of CBD.

Also, research has shown similar activity of CBD against gram-positive bacteria (e.g. Staphyloccocus aureaus and Streptococcus pneumoniae) compared to conventional antibiotics (e.g. vancomycin), further explaining the role of CBD in bacterial infections.

Furthermore, the specific combination of THC and CBD used in combination therapies is shown to have synergistical properties and, for example, suppress neuroinflammation and reduce muscle spasticity. Also, CBD or THC alone as well as combinations are shown to have anti-inflammatory and anti-hyperalgesia effects. Furthermore, research shows that both CBD alone or CBD in combination with THC may alter fear memory and may have positive effects on anxiety in the case of post-traumatic stress syndrome.

The combination of THC and CBD has been shown to be efficient in the treatment of neuropathic pain, for example allodynia. In this case, CBD has shown to enhance the pain-relieving actions of THC.

As used herein and unless otherwise specified, the term “allodynia” is to be understood as a central pain sensitization following from stimulations which are normally not painful. Often, these stimulations are repetitive.

Pain and inflammation can be regarded as physiological responses of the human body to e.g. tissue injury and infection. Two response phases can be distinguished: acute and chronic. The acute phase being an early, non-specific phase which is characterized by local vasodilatation and an increased capillary permeability, an accumulation of fluid and proteins in the interstitial spaces, the migration of neutrophils away from the capillaries and the release of inflammatory mediators such as cytokines. The release of said pro-inflammatory mediators leads among others to a perception by the human body which is defined as “pain”.

Chronic pathological pain endures beyond the resolution of the pain source and can even deeply impact the quality of life of people suffering therefrom. In some embodiments, the pain may be neuropathic pain. Such neuropathic pain is to be understood as pain due to peripheral or central nervous system injury, wherein the sensitization of pain is generally evoked by sensory stimuli in the absence of noxious stimuli.

In some embodiments, the combination as defined herein, or at least one of the cannabinoids of the combination may have at least one property selected from the list comprising: anti-inflammatory, analgesic, antipruritic, antifungal or antibacterial agent properties.

In another embodiment, the present invention provides a combination as defined herein for use in the treatment of a disorder selected from the list comprising: pain, blisters, neurodegenerative disorders or post-traumatic stress syndrome is disclosed, wherein said use includes the reduction of opioid consumption/dependency in the treatment of said disorders.

The current combination provides for a suitable alternative for medicines containing for example opioid substances. As these opioid substances are shown to entail a substantial risk of addiction and may result in fatal overdoses, the current combination offers an alternative fulfilling a long-term need, more specifically in this area of treatment. The current combination reduces the risk of drug addiction (e.g. opioid addiction) especially when long-term treatments are necessary. The longer the duration of drug use such as morphine, oxycodone or other strong analgesics, the higher the risk of addiction. Also, the longer these analgesics are used, the higher the doses because of substantive amount of habituation. This may contribute to the risk of a fatal overdose. Therefore, the use of the current combination provides for a suitable alternative having a positive effect of reducing the risk of drug addiction compared to the use of conventional drugs (e.g. opioids).

Finally, the anti-hyperalgesia effects as shown for the combination of THC and CBD may offer a valuable treatment for opioid-induced hyperalgesia which is associated with long-term use of opioids such as morphine, oxycodone and methadone.

In another embodiment, the combination for use as defined herein may comprise one or more additional pharmaceutically active agents suitable for use in the treatment of said disorders.

In some embodiments, the additional pharmaceutically active agents may act as ligands of the bodily cannabinoid receptors (e.g. cannabinoid receptor type 1 (CB₁), cannabinoid receptor type 2 (CB₂)) and other cannabinoid receptors.

In some embodiments, the uptake of the combination as defined herein, or at least one of the cannabinoids of the combination may happen via the oromucosal route.

In other embodiments, the additional pharmaceutically active agents may be drug classes which contribute to the reduction of secondary symptoms such as pain and inflammation.

### EXAMPLES

Example 1: Production of transdermal nanofibrous film with 10 mg Δ9-THC as active pharmaceutical ingredient (API) using electrospinning.

Phase 1: Poly(vinyl alcohol) (PVA) was dissolved in distilled water at an ambient temperature 20° C. with a polymer concentration of 0.25 g/ml. The solution was magnetically stirred for at least 4 h at room temperature in order to obtain homogenously dissolved solutions. The solutions were degassed without stirring for 1 h prior to electrospinning processing. To ensure the uniformity of the drug content in the electrospun nanofibers, 10 mg Δ9-THC was first dissolved, after which the polymer was added to prepare the electrospinning solution. Mechanical stirring was applied for at least 20 min at room temperature to obtain homogeneous solutions.

Phase 2: The spinning solutions were carefully placed into a plastic syringe (5 mL), with great care taken to avoid any air bubbles. A metal dispensing tip (spinneret; gauge 20, 0.61 mm inner diameter) was attached to the syringe. The positive electrode of a high voltage power DC supply was then connected to the spinneret. The grounded electrode was connected to a metal collector (17×17 cm2) wrapped with aluminum foil. Electrospinning was carried out under ambient conditions (22° C. and relative humidity 35%). An electrical potential of 15 kV was applied across a fixed distance of 12 cm between the spinneret and the collector. The polymer solution was dispensed from the syringe at a feed rate of 1.2 mL/h using a syringe pump. Fibers were stored in a vacuum desiccator post-synthesis to facilitate the removal of residual organic solvents and moisture. The as-spun nanofibrous membranes were carefully peeled from the aluminum foils and cut into 1 cm×1 cm pieces for the pharmacotechnical evaluations.

Example 2: Production of nano-encapsulated cannabinoid drug nanoparticles (dry powder) with 1% wt/wt Δ9-THC (10 mg/mL) via electro-hydrodynamic atomization (EHDA) (also referred to as “electrospraying”) to improve hydrophilicity of Δ9-THC and transdermal absorption in transdermal nanofibrous films produced via electrospinning. To improve bioavailability and stability of Δ9-THC nanoencapsulation via electrospraying was used. This involves entrapment of the API in a matrix at the size range of nanometers. Amongst other advantages, greater surface area of nanoparticles provide improved dispersion of APIs, in turn enhancing its bioavailability. Briefly, formulations were processed by electrospraying using a Fluidnatek® LE10 lab line from Bioinicia S. L. (Valencia, Spain) with a variable high-voltage 0-30 kV power supply. Formulations were fed into a 5-mL syringe and pumped for each solution through a stainless-steel needle injector. Samples were collected on a grounded metallic flat plate. The applied voltage, flow-rate, and tip-to-collector distance were optimized based on visual observation of the Taylor cone formation and no droplet deposition on the collector. Different biopolymers from different origins were evaluated as potentials matrices such as Whey protein concentrate (WPC) with a protein content of 80%, polysaccharide maltodextrin, vegetal protein zein or plastic derived polymer polyvinylpyrrolidone (PVP). Surfactant was added to all solutions at a concentration of 6 wt. % with respect to the polymer weight in order to improve its sprayability according to previous authors. The nano-encapsulated drug nanoparticles (dry powder) were then incorporated into transdermal nanofibrous films via electrospinning as described in example 1.

Example 3: Production of transdermal nanofibrous film with 10 mg Δ9-THC as active pharmaceutical ingredient (API) using electrospinning.

Phase 1: Polyethylene Oxide (PEO) was dissolved in distilled water at an ambient temperature 20° C. with a polymer concentration of 8%. The solution was magnetically stirred for at least 4 h at room temperature in order to obtain homogenously dissolved solutions. The solutions were degassed without stirring for 1 h prior to electrospinning processing. To ensure the uniformity of the drug content in the electrospun nanofibers, a mixture of 10 mg Δ9-THC/sesame oil:water 20:80 (w/w) was first dissolved in Chloroform:Methanol (80:20) after which the polymer was added. Additionally, a permeation enhancer (SPAN, 1 wt %) and antioxidant (Ascorbic acid, 1 wt %) were added to prepare the electrospinning solution. Mechanical stirring (e.g. ultraturrax) together with sonication was applied for at least 20 min at room temperature to obtain homogeneous solutions. The mixture (CNV-6) was incorporated into a transdermal nanofibrous form via electrospinning as described in example 1.

Two other mixtures (CNV-11 and CNV-12) were prepared in the same way as CNV-6 but the permeation enhancer was replaced by 1% oleic acid and the antioxidant by 1% TGPS (CNV-11) and for CNV-12 solely the antioxidant was replaced by 1% TGPS.

A Franz diffusion cell was used to test the in vitro permeation. A Franz cell consists of a donor and acceptor chamber which is separated by a diffusion membrane (human skin or Strat-M® membrane) predict human skin diffusion. The donor chamber contained the mixture whereas the acceptor chamber contained aqueous buffer maintained at 32 or 37° C. with constant stirring. An intimate contact of membrane to both phases allowed for diffusion of active from donor chamber to acceptor chamber.

Strat-M membrane is a synthetic, non-animal based model for transdermal diffusion testing. Strat-M™ (Merck Millipore, Burlington, Mass., USA) was composed of multiple layers of polyester sulfone. Like human skin, the Strat-M™ membrane has multiple layers with varied diffusivity. The outer layer consists of two layers of polyethersulfone (PES, more resistant to diffusion), while the bottom layer is a more diffusive polyolefin layer.

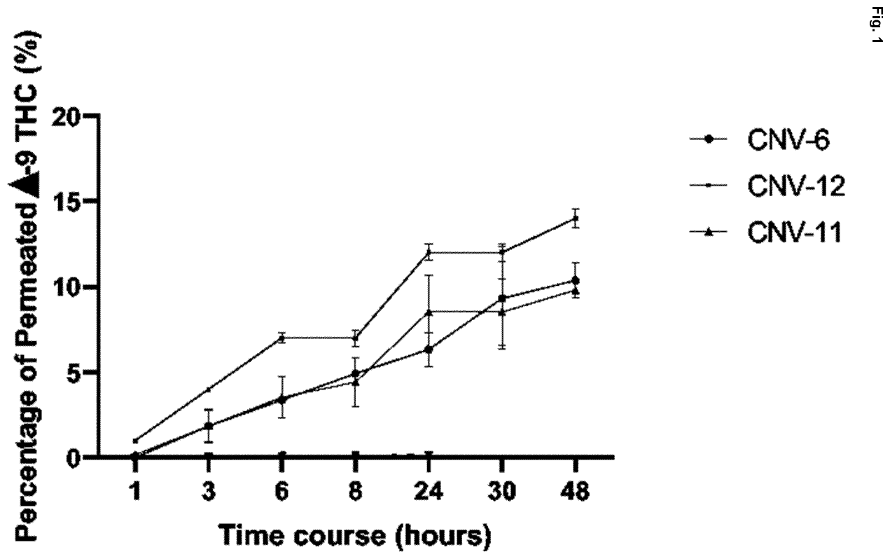

In vitro permeability testing of the three mixtures (CNV6, CNV-11, CNV-12) resulted in a permeability ranging from 9-13% after 48 hours (FIG. 1). While CNV-6 and CNV-11 have a permeability of about 10%, the mixture CNV-12 performs better with a permeability of about 13%.

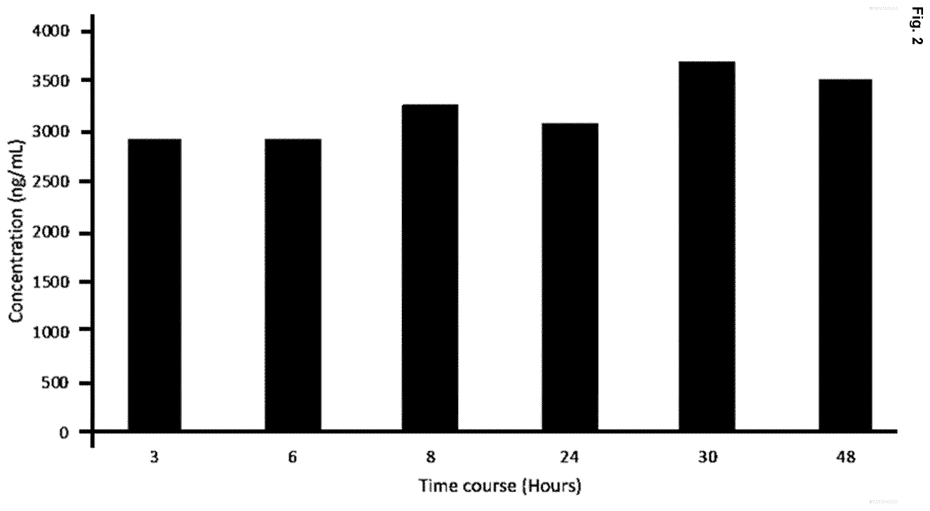

The THC of CNV-11 does not seem to degrade in the receiver solution EtOH:H₂O (50/50 vol) over a time of 48 hours (FIG. 2) and thus shows a good stability of THC in the film.

1. A combination comprising Tetrahydrocannabinol (THC) and Cannabidiol (CBD) for use in the treatment of a disorder selected from the list comprising: pain, blisters, neurodegenerative disorders or post-traumatic stress syndrome; wherein said combination is formulated in a transdermal patch,
characterized in that said transdermal patch comprises at least one transdermal nanofibrous electrospun film.

2. The combination for use according to claim 1, wherein said film comprises nano-encapsulated THC and/or CBD.

3. The combination for use according to anyone of claim 1 or 2, wherein at least one of said THC and CBD is a prodrug.

4. The combination for use according to anyone of claims 1 to 3, wherein said transdermal patch is selected from the list comprising: reservoir patch, matrix patch, vapor patch or drug-in-adhesive patch.

5. The combination for use according to claim 4, wherein said drug-in-adhesive patch is selected from the list comprising: single-layer drug-in-adhesive patch or multi-layer drug-in-adhesive patch.

6. The combination for use according to anyone of claims 1 to 5, wherein said transdermal patch and/or film further comprises at least one polymer.

7. The combination for use according to claim 6, wherein said polymer is polyethylene oxide.

8. The combination for use according to anyone of claims 1 to 7, wherein said transdermal patch and/or film further comprises a skin penetration enhancer.

9. The combination for use according to anyone of claims 1 to 8, wherein said transdermal patch and/or film further comprises an antioxidant.

10. The combination for use according to anyone of claims 1 to 9, wherein said blisters are caused by burns or other related traumas or a disorder selected from the list comprising:
skin allergies, different forms of eczema, bullous pemphigoid, bullous impetigo, dermatitis herpetiformis, pemphigus vulgaris, mucous membrane pemphigoid, pemphigoid gestationis, epidermolysis bullosa, pemphigus foliaceous or toxic epidermal necrolysis.

11. The combination for use according to anyone of claims 1 to 10, wherein said neurodegenerative disorder is selected from the list comprising: Alzheimer's disease (AD) and other dementias, Parkinson's disease (PD) and PD-related disorders, Essential Tremor, Multiple System Atrophy, Huntington's disease or Motor Neuron Diseases (MND).

12. The combination for use according to anyone of claims 1 to 11, wherein said use includes the alleviation of secondary symptoms caused by said disorders.

13. The combination for use according to claim 12 wherein said secondary symptoms are selected from the list comprising, secondary pain, itch, secondary impetigos, swelling, inflammation or bacterial infection.

14. The combination for use according to anyone of claims 1 to 13, wherein said use includes the reduction of opioid consumption/dependency in the treatment of at least one of said disorders.

15. The combination for use according to anyone of claims 1 to 14, further comprising one or more additional pharmaceutically active agents suitable for use in the treatment of said disorders.
